(19) United States
(12) Patent Application Publication
(10) Pub. No.: US 2023/0273205 A1
(43) Pub. Date: Aug. 31, 2023

(54) Methods and Compositions for Diagnosing and Treating Cancer

(71) Applicant: La Jolla Institute for Immunology, La Jolla, CA (US)

(72) Inventors: Catherine Lynn Hedrick, La Jolla, CA (US); Melissa Meyer, La Jolla, CA (US)

(21) Appl. No.: 18/016,538

(22) PCT Filed: Jul. 14, 2021
(86) PCT No.: PCT/US2021/041604

§ 371 Date: Jan. 17, 2023

Related U.S. Application Data
(60) Provisional application No. 63/051,882, filed on Jul. 14, 2020.

(51) Int. Cl. G01N 33/569; G01N 33/574; A61K 35/17

(52) U.S. Cl. CPC G01N 33/56972; G01N 33/574; A61K 35/17; G01N 2333/705

(57) ABSTRACT

The present invention includes methods for detecting B-cell like neutrophils (“BNeuts”) comprising: obtaining a biological sample from a subject; and detecting whether BNeuts are present or increased in the biological sample by contacting the biological sample with an agent capable of detecting CD3− CD56−CD79b⁺CD66b⁺ PAX5⁺CD19− cells, CD3−CD56−CD79a⁺CD79b⁺CD66b⁺ PAX5⁺CD19− cells, or both, and detecting the increase of BNeuts in the biological sample.

### CROSS-REFERENCE TO RELATED APPLICATIONS

This application claims priority to U.S. Provisional Application Ser. No. 63/051,882, filed Jul. 14, 2020, the entire contents of which are incorporated herein by reference.

### FIELD OF THE INVENTION

The present invention relates in general to the field of cancer immunotherapy, and more particularly, to the use of neutrophils expressing B cell receptors (“BNeuts”) as a biomarker informing the diagnosis and/or presence of cancer. It moreover pertains to methods of using these cells for the treatment of cancer.

### BACKGROUND OF THE INVENTION

Neutrophils are innate immune cells that are early responders to infection and inflammation. Historically, neutrophils were described as a relatively homogenous, but recent studies show that neutrophils display phenotypic and functional heterogeneity. Neutrophils have been shown to play both pro-tumoral and anti-tumoral roles but exactly which neutrophil subsets are pro-tumoral vs anti-tumoral is still unclear. To better understand and target neutrophil functions in cancer, the inventors have examined neutrophil heterogeneity and how it changes from health to disease.

### SUMMARY OF THE INVENTION

Traditionally, neutrophils have been studied as a single population and neutrophil heterogeneity has not previously been completely appreciated. Herein is described a newly defined neutrophil subset with a B cell signature. This population is absent from the blood of healthy individuals but found in the blood of cancer patients at early stage. The appearance of BNeuts in the blood of cancer patients is novel and understanding the function of this neutrophil is important for new therapeutic approaches targeting neutrophils.

The invention is based, in part, on the role of neutrophils in immune responses. As broadly described herein, a method of detecting B-cell like neutrophils (“BNeuts”) is provided, comprising: obtaining a biological sample from a subject; and detecting whether BNeuts are present or increased in the biological sample by contacting the biological sample with one or more agents capable of detecting CD3−CD56−CD79b⁺CD66b⁺PAX5⁺CD19− cells, CD3− CD56−CD79a⁺CD79b⁺CD66b⁺PAX5⁺CD19− cells, or both.

In other embodiments, the method comprises detecting the presence or an elevated level of expression of at least one of: CD40, CD80, CD86, HLA-DR on the BNeuts. In some embodiments, the step of detecting comprises measuring mRNA, protein, or both.

In certain embodiments, the biological sample is a blood sample. In other embodiments, the biological sample is obtained from a subject suspected of having cancer. In some embodiments, the cancer is at an early stage. In other embodiments, the cancer is selected from a colorectal, a melanoma, a lung, a liver, a head and neck, or a breast cancer issue.

In some embodiments, the presence or increase of BNeuts is detected in the biological sample as compared to a healthy subject. In alternative embodiments, the one or more agents is an antibody, a small molecule, a peptide mimetic, an aptamer, a nucleic acid, or combinations thereof.

As broadly described herein, a method of diagnosing and treating a cancer in a patient is provided, the method comprising the steps of determining whether the patient has or has an increase B-cell like neutrophils (BNeut) cells by: obtaining or having obtained a biological sample from the patient; performing or having performed an assay on the biological sample to determine if the patient has or has an increase in BNeuts, wherein the BNeuts are CD3− CD56−CD79b⁺CD66b⁺PAX5⁺CD19− cells, CD3− CD56−CD79a⁺CD79b⁺CD66b⁺PAX5⁺CD19− cells, or both, when compared to a reference level generated from a healthy patient; wherein if the patient has BNeuts or shows an increase in BNeuts, which indicates the presence of cancer, administering a cancer treatment or therapy to the patient.

In certain embodiments, the presence of BNeuts is determined from a blood sample. In other embodiments, the step of detecting is measuring mRNA, protein, or both. In some embodiments, the cancer is an early-stage cancer. In still other embodiments, the cancer is selected from a colorectal, a melanoma, a lung, a liver, a head and neck, and a breast cancer.

In certain embodiments, further comprising detecting if the BNeuts express one or more of the following markers: CD40, CD80, CD86, HLA-DR.

As broadly described herein, a method for treating a patient suffering from a cancer is provided, the method comprising the steps of determining whether the patient has B-cell like neutrophils (BNeut) or an increase in BNeuts, when compared to a reference level generated for a healthy patient by: obtaining or having obtained a biological sample from the patient; and performing or having performed an assay on the biological sample to determine if the patient has BNeuts; and if the patient has BNeuts, then administering a cancer treatment or therapy to the patient.

In certain embodiments, the presence of BNeuts is determined from blood sample. In other embodiments, the step of detecting is measuring mRNA, protein, or both. In some embodiments, the BNeuts are CD3− CD56−CD79b⁺CD66b⁺PAX5⁺CD19− cells, CD3− CD56−CD79a⁺CD79b⁺CD66b⁺PAX5⁺CD19− cells or both.

In some embodiments, the cancer is an early-stage cancer. In other embodiments, the cancer is selected from a colorectal, a melanoma, a lung, a liver, a head and neck, and a breast cancer. In still other embodiments, the BNeuts express or have an elevated level of expression one or more of the following markers: CD40, CD80, CD86, or HLA-DR.

In certain embodiments, the cancer treatment or therapy comprises surgery, chemotherapy, radiation therapy, or immunotherapy. In other embodiments, the cancer immunotherapy comprises adoptive cell therapy. In some embodiments, the adoptive cell therapy comprises administering a population of BNeuts, CD3− CD56−CD79b⁺CD66b⁺PAX5⁺CD19− cells, CD3− CD56− CD79a⁺CD79b⁺CD66b⁺PAX5⁺CD19− cells, or any combination thereof. In certain embodiments, the adoptive cell therapy comprises administering a population of engineered cells.

As broadly described herein, a method for treating a cancer patient is provided, the method comprising the steps of: determining whether the cancer patient has an increase in B-cell like neutrophils (“BNeuts”) by: obtaining or having obtained a biological sample from the patient; performing or having performed an assay on the biological sample to determine if the patient has an increase in BNeuts when compared to a baseline level of BNeuts present in a healthy patient; and if the patient has an increase in BNeuts when compared to a baseline level of BNeuts present in the healthy patient, then providing an adoptive cell therapy that comprises BNeuts, CD3−CD56−CD79b⁺CD66b⁺PAX5⁺CD19− cells, CD3− CD56−CD79a⁺CD79b⁺CD66b⁺PAX5⁺CD19− cells, or any combination thereof, to the patient in an amount sufficient to reduce or treat the cancer.

In certain embodiments, the cancer is an early-stage cancer. In other embodiments, the cancer is selected from a colorectal, a melanoma, a lung, a liver, a head and neck, and a breast cancer.

In certain embodiments, the method further comprising the step of isolating BNeuts from the cancer patient, growing the BNeuts ex vivo, and isolating the BNeuts prior to the adoptive cell therapy. In other embodiments, BNeuts are autologous, syngeneic, allogeneic, or xenogeneic.

As broadly described herein, a kit for cell sorting or detecting B-cell like neutrophils (“BNeuts”) from a biological sample, the kit comprising detecting agents for at least one of: CD3−CD56− CD79b⁺CD66b⁺ PAX5⁺CD19− cells, CD3− CD56−CD79a⁺CD79b⁺CD66b⁺PAX5⁺CD19− cells, or both.

As broadly described herein, a method for treating a patient is provided, the method comprising the steps of: identifying that the patient is in need of a treatment for cancer; and providing an adoptive cell therapy that comprises BNeuts, CD3−CD56−CD79b⁺CD66b⁺PAX5⁺CD19− cells, CD3−CD56− CD79a⁺CD79b⁺CD66b⁺PAX5⁺CD19− cells, or any combination thereof, to the patient in an amount sufficient to reduce or treat the cancer, wherein the BNeuts are autologous, syngeneic, allogeneic, or xenogeneic.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with any accompanying Figures.

### BRIEF DESCRIPTION OF THE FIGURES

For a more complete understanding of the features and advantages of the present invention, reference is now made to the detailed description of the invention along with the accompanying figures and in which:

FIGS. 1A and 1B show the identification of immature neutrophils with B cell features, namely BNeuts, in healthy human bone. FIG. 1A) 10× scRNA-seq of human bone marrow Lin−CD66b⁺CD117+ immature neutrophils was used to identify a new neutrophils subset that expresses B cell genes. Cells were clustered using Louvain community (Seurat) and plotted using UMaP. FIG. 1B) Differential gene expression between BNeuts and CD79b− immature neutrophils shows increased BNeut expression of a number of B cell genes.

FIGS. 2A to 2E show that bone marrow BNeuts maintain neutrophil phenotypes and derive from neutrophils progenitors. Analysis of healthy human bone marrow BNeuts (see FIG. 3D), neutrophils (CD3−CD56−CD19−CD203c−Siglec8−CD66b+CD79b−), and B cells (CD3−CD56−CD66b−CD19+). FIG. 2A) Cytospins show BNeuts have a large cytoplasm, similar to neutrophils. FIG. 2B) Flow cytometry shows BNeuts maintain a high side scatter, like neutrophils, and express equivalent levels of CD79b to B cells. FIG. 2C-FIG. 2D) Bulk RNA-seq shows BNeuts cluster closer to neutrophils than B cells in MDS analysis which is reflected in the heatmap. FIG. 2E) Human early neutrophil progenitors that are restricted to the neutrophil lineage were transferred into NSG-SGM3 mice. After 5 days mouse bone marrow was analyzed for human BNeuts. CD79b+ BNeuts, indicated in red, show BNeuts can derive from neutrophil progenitors.

FIGS. 3A to 3G shows that BNeuts are found in the periphery of cancer patients and maintain neutrophil functions. Melanoma patient blood neutrophils analyzed by mass cytometry reveals CD79b+ BNeuts. FIG. 3A) Mass cytometry data projected into a UMaP. BNeuts highlighted in red identified by CD79b expression. FIG. 3B) Heatmap reveals unique protein expression pattern in BNeuts compared to neutrophils (CD79b−). FIG. 3C) Image stream of melanoma patient blood BNeuts, neutrophils (CD79b−), and B cells shows BNeuts are single cells that co-express CD79b and CD66b. FIG. 3D) BNeuts quantitation in the blood by flow cytometry reveals BNeuts are enriched in the periphery of melanoma patients. FIG. 3E) BNeuts identified in publicly available scRNA-seq data of head and neck tumor infiltrating neutrophils by CD79b and CD79a expression (Cillo et al., 2020). FIG. 3F) Netosis measured in BNeuts and neutrophils (CD79b−) from melanoma patient blood by flow cytometry. Whole blood was incubated with and without PMA. NETs identified as MPO+cit-H3+. FIG. 3G) Antibody production measured by cell surface expression of IgM (and IgD-data not shown) indicates BNeuts do not produce antibodies and present them on the cell surface. * p<0.05, ** p<0.01, **** p<0.0001 by unpaired T test.

FIGS. 4A to 4D shows that BNeuts express an antigen presentation program to modulate CD4+ T Cells. FIG. 4A) Antigen presentation machinery measured by flow cytometry in BNeut, neutrophils (CD79b−), and B cells from melanoma patient blood shows elevated expression of CD40, CD80, and HLA-DR in BNeuts compared to neutrophils (CD79b−). FIG. 4B) Antigen uptake is increased in BNeuts compared to neutrophils (CD79b−) from melanoma patient blood. Whole blood was co-cultured with either zymosan (top graph) or ZsGreen+ tumor cells (bottom graph). Zymosan/ZsGreen uptake was measured by flow cytometry. FIG. 4C) BNeuts are able to stimulate CD4+ T cell viral memory responses. BNeuts or neutrophils (CD79b−) were isolated from melanoma patient blood and loaded with MHCII restricted viral peptides. After, BNeuts or neutrophils (CD79b−) were co-culture with healthy donor T cells and CD4+ T cells IFNy expression was measured by flow cytometry, as an indication of activation. FIG. 4D) BNeuts are enriched in melanoma patient blood in stage I and II. BNeuts and neutrophils (CD79b−) were measured in melanoma patient blood and healthy donors by flow cytometry. * p<0.05, ** p<0.01, *** p<0.001, **** p<0.0001 by unpaired T test.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

In some embodiments described herein is a population of B cell-like neutrophils, termed BNeuts; a new neutrophil subset restricted to the bone marrow of healthy individuals but found in the blood of melanoma patients. BNeuts maintain a neutrophil program while upregulating a number of B cell factors, including Pax5 and antigen presentation machinery allowing them to modulate CD4+ T cell responses.

The inventors employed single-cell transcriptome technology to profile immature neutrophils from bone marrow of healthy individuals. A new neutrophil subset that expresses several B cell markers, including CD79a/b, a component of the B cell receptors and PAX5, a lineage-specific transcriptional factor, were identified and named “BNeuts”. Using flow cytometry, the inventors found BNeuts are confined to the bone marrow of healthy individuals but expand into the blood of melanoma patients at an early stage, suggesting BNeuts might be a biomarker for disease. Further, BNeuts were in the mouse models of breast and lung cancer. The BNeut appears and acts more like a neutrophil than B cell with a neutrophil-like transcriptional profile, large cytoplasm, ROS production, and ability to produce NETs. Using imaging, it is shown that BNeuts are single cells co-expressing both CD66b and CD79b, in the absence of the B cell surface marker CD19. To understand why neutrophils might use a B cell program, different B cell functions were explored, including antigen presentation, a function that is usually absent from neutrophils. BNeuts are excellent at phagocytosis, including uptake of tumor cells. Further, BNeuts have increased expression of antigen presentation related genes compared to other neutrophils and can modulate CD4+ T cell responses. This disclosure identifies a novel neutrophil subset expressing B cell markers that is preferentially expanded in the blood of melanoma patients and is poised to control T cell responses during disease.

As broadly described herein, a method of detecting B-cell like neutrophils (“BNeuts”) is provided, comprising: obtaining a biological sample from a subject; and detecting whether BNeuts are present or increased in the biological sample by contacting the biological sample with an agent capable of detecting CD3⁻CD56⁻CD79b⁺CD66b⁺PAX5⁺CD19⁻ cells, CD3⁻CD56⁻CD79a⁺CD79b⁺CD66b⁺PAX5⁺CD19⁻ cells, or both, and detecting the increase of BNeuts in the biological sample.

In other embodiments, the method comprises detecting the presence or an elevated level of expression of at least one of: CD40, CD80, CD86, HLA-DR on the BNeuts. In some embodiments, the step of detecting comprises measuring mRNA, protein, or both.

In certain embodiments, the biological sample is a blood sample. In other embodiments, the biological sample is obtained from a subject suspected of having cancer. In some embodiments, the cancer is at an early stage. In other embodiments, the cancer is selected from a colorectal, a melanoma, a lung, a liver, a head and neck, or a breast cancer issue.

In some embodiments, the presence or increase of BNeuts is detected in the biological sample as compared to a healthy subject. In alternative embodiments, one or more agents used to detect the presence or increase of BNeuts is an antibody, a small molecule, a peptide mimetic, an aptamer, a nucleic acid, or combinations thereof.

In certain embodiments of the present invention an agonist or antagonist is an antibody, a small molecule, a protein, a peptide, an antisense nucleic acid or an aptamer, including an antibody-small molecule conjugate, a bispecific antibody or bispecific molecule.

As broadly described herein, a method of diagnosing and treating a cancer in a patient is provided, the method comprising the steps of determining whether the patient has or has an increase B-cell like neutrophils (BNeut) cells by: obtaining or having obtained a biological sample from the patient; performing or having performed an assay on the biological sample to determine if the patient has or has an increase in BNeuts, wherein the BNeuts are CD3− CD56−CD79b⁺CD66b⁺PAX5⁺CD19− cells, CD3− CD56−CD79a⁺CD79b⁺CD66b⁺PAX5⁺CD19− cells, or both, when compared to a reference level generated a healthy patient; wherein if the patient has BNeuts or shows an increase in BNeuts, it indicates the presence of cancer; and subsequently administering a cancer treatment or therapy to the patient.

In certain embodiments, the presence of BNeuts is determined from a blood sample. In other embodiments, the step of detecting is measuring mRNA, protein, or both. In some embodiments, the cancer is an early-stage cancer. In still other embodiments, the cancer is selected from a colorectal, a melanoma, a lung, a liver, a head and neck, and a breast cancer.

In certain embodiments, the BNeuts express one or more of the following markers: CD40, CD80, CD86, HLA-DR.

As broadly described herein, a method for treating a patient suffering from a cancer is provided, the method comprising the steps of determining whether the patient has B-cell like neutrophils (BNeut), when compared to a reference level generated for a healthy patient by: obtaining or having obtained a biological sample from the patient; and performing or having performed an assay on the biological sample to determine if the patient has BNeuts; and if the patient has BNeuts, then administering a cancer treatment or therapy to the patient.

In certain embodiments, the presence of BNeuts is determined from blood sample. In other embodiments, the step of detecting is measuring mRNA, protein, or both. In some embodiments, the BNeuts are CD3− CD56−CD79b⁺CD66b⁺ PAX5⁺CD19− cells, CD3− CD56−CD79a⁺CD79b⁺CD66b⁺ PAX5⁺CD19− cells or both.

In some embodiments, the cancer is an early-stage cancer. In other embodiments, the cancer is selected from a colorectal, a melanoma, a lung, a liver, a head and neck, and a breast cancer. In still other embodiments, the BNeuts express or have an elevated level of expression one or more of the following markers: CD40, CD80, CD86, or HLA-DR.

In certain embodiments, the cancer treatment or therapy comprises surgery, chemotherapy, radiation therapy, or immunotherapy. In other embodiments, the cancer immunotherapy comprises adoptive cell therapy. In some embodiments, the adoptive cell therapy comprises administering a population of BNeuts, CD3− CD56−CD79b⁺CD66b⁺PAX5⁺CD19− cells, CD3− CD56− CD79a⁺CD79b⁺CD66b⁺PAX5⁺CD19− cells, or any combination thereof. In certain embodiments, the adoptive cell therapy comprises administering a population of engineered cells.

As broadly described herein, a method for treating a cancer patient is provided, the method comprising the steps of: determining whether the cancer patient has an increase in B-cell like neutrophils (“BNeuts”) by: obtaining or having obtained a biological sample from the patient; performing or having performed an assay on the biological sample to determine if the patient has an increase in BNeuts when compared to a baseline level of BNeuts present in a healthy patient; and if the patient has an increase in BNeuts when compared to a baseline level of BNeuts present in the healthy patient, then providing an adoptive cell therapy that comprises BNeuts, CD3−CD56−CD79b⁺CD66b⁺PAX5⁺CD19− cells, CD3− CD56−CD79a⁺CD79b⁺CD66b⁺PAX5⁺CD19− cells, or any combination thereof, to the patient in an amount sufficient to reduce or treat the cancer.

In certain embodiments, the cancer is an early-stage cancer. In other embodiments, the cancer is selected from a colorectal, a melanoma, a lung, a liver, a head and neck, and a breast cancer.

In certain embodiments, the method further comprising the step of isolating BNeuts from the cancer patient, growing the BNeuts ex vivo, and isolating the BNeuts prior to the adoptive cell therapy. In other embodiments, BNeuts are autologous, syngeneic, allogeneic, or xenogeneic.

As broadly described herein, a kit for cell sorting or detecting B-cell like neutrophils (“BNeuts”) from a biological sample, the kit comprising detecting agents for at least one of: CD3−CD56− CD79b⁺CD66b⁺ PAX5⁺CD19− cells, CD3− CD56−CD79a⁺CD79b⁺CD66b⁺PAX5⁺CD19− cells, or both.

As broadly described herein, a method for treating a patient is provided, the method comprising the steps of: identifying that the patient is in need of a treatment for cancer; and providing an adoptive cell therapy that comprises BNeuts, CD3−CD56−CD79b⁺CD66b⁺PAX5⁺CD19− cells, CD3−CD56− CD79a⁺CD79b⁺CD66b⁺PAX5⁺CD19− cells, or any combination thereof, to the patient in an amount sufficient to reduce or treat the cancer, wherein the BNeuts are autologous, syngeneic, allogeneic, or xenogeneic.

In certain embodiments of the present invention an agonist or antagonist is an antibody, a small molecule, a protein, a peptide, an antisense nucleic acid or an aptamer, including an antibody-small molecule conjugate, a bispecific antibody or bispecific molecule.

As used herein, an “antibody” refers to a polyclonal or monoclonal antibody, or binding fragment thereof. Antibodies sometimes are IgG, IgM, IgA, IgE, or an isotype thereof (e.g., IgG1, IgG2a, IgG2b or IgG3), sometimes are polyclonal or monoclonal, and sometimes are chimeric, humanized or bispecific versions of an antibody. In some embodiments an antibody or portion thereof, comprises a chimeric antibody, Fab, Fab′, F(ab′)2, Fv fragment, scFv, diabody, aptamer, synbody, camelid, the like and/or a combination thereof.

Methods of the invention include treatment methods, which result in any therapeutic or beneficial effect. As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis. The cancer can be an early stage. Examples of cancers include: a colorectal, a melanoma, a lung, a liver, a head and neck, or a breast cancer issue

As used herein, “anti-tumor immunity” in a subject refers to reducing or preventing the symptoms or cancer from occurring in a subject that is predisposed or does not yet display symptoms of the cancer. The anti-tumor immunity includes B-cell like neutrophils (“BNeuts”), including: CD3− CD56−CD79b⁺CD66b⁺PAX5⁺CD19− cells, CD3− CD56−CD79a⁺CD79b⁺CD66b⁺PAX5⁺CD19− cells, or both. The BNeuts can also express at least one of: CD40, CD80, CD86, or HLA-DR. For treatment, the BNeuts can be provided in the form of adoptive cell therapy and can include engineered cells and/or cells grown ex vivo, such as autologous, syngeneic, allogeneic, or xenogeneic cells.

In some embodiments a subject is in need of a treatment, cell or composition described herein. In certain embodiments a subject has or is suspected of having a neoplastic disorder, neoplasia, tumor, malignancy or cancer. In some embodiments a subject in need of a treatment, cell or composition described herein has or is suspected of having a neoplastic disorder, neoplasia, tumor, malignancy or cancer. In certain embodiments an engineered T cell described herein is used to treat a subject having, or suspected of having, a neoplastic disorder, neoplasia, tumor, malignancy or cancer.

In some embodiments, presented herein is a method of treating a subject having or suspected of having, a neoplasia, neoplastic disorder, tumor, cancer, or malignancy. In certain embodiments, a method of treating a subject comprises administering a therapeutically effective amount of engineered BNeuts to trigger or control T cell immunity in the subject. In certain embodiments, a method comprises reducing or inhibiting proliferation of a neoplastic cell, tumor, cancer or malignant cell, comprising contacting the cell, tumor, cancer or malignant cell, with the engineered BNeuts in an amount sufficient to reduce or inhibit proliferation of the neoplastic cell, tumor, cancer or malignant cell.

In some embodiments, a method of reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy, comprises administering to a subject an amount of an engineered BNeuts sufficient to reduce or inhibit metastasis of the neoplasia, tumor, cancer or malignancy to other sites, or formation or establishment of metastatic neoplasia, tumor, cancer or malignancy at other sites distal from the primary neoplasia, tumor, cancer or malignancy.

Non-limiting examples of a neoplasia, neoplastic disorder, tumor, cancer or malignancy include a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma. A neoplasia, neoplastic disorder, tumor, cancer or malignancy may comprise or involve hematopoietic cells. Non-limiting examples of a sarcoma include a lymphosarcoma, liposarcoma, osteosarcoma, chondrosarcoma, leiomyosarcoma, rhabdomyosarcoma or fibrosarcoma. In some embodiments, a neoplasia, neoplastic disorder, tumor, cancer or malignancy is a myeloma, lymphoma or leukemia. In some embodiments, a neoplasia, neoplastic disorder, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer. In some embodiments, a neoplasia, neoplastic disorder, tumor, cancer or malignancy comprises a small cell lung or non-small cell lung cancer. In some embodiments, a neoplasia, neoplastic disorder, tumor, cancer or malignancy comprises a stem cell neoplasia, tumor, cancer or malignancy. In some embodiments, a neoplasia, neoplastic disorder, tumor, cancer or malignancy.

In some embodiments, a method inhibits, or reduces relapse or progression of the neoplasia, neoplastic disorder, tumor, cancer or malignancy. In some embodiments, a method comprises administering an anti-cell proliferative, anti-neoplastic, anti-tumor, anti-cancer or immune-enhancing treatment or therapy. In some embodiments, a method of treatment results in partial or complete destruction of the neoplastic, tumor, cancer or malignant cell mass; a reduction in volume, size or numbers of cells of the neoplastic, tumor, cancer or malignant cell mass; stimulating, inducing or increasing neoplastic, tumor, cancer or malignant cell necrosis, lysis or apoptosis; reducing neoplasia, tumor, cancer or malignancy cell mass; inhibiting or preventing progression or an increase in neoplasia, tumor, cancer or malignancy volume, mass, size or cell numbers; or prolonging lifespan. In some embodiments, a method of treatment results in reducing or decreasing severity, duration or frequency of an adverse symptom or complication associated with or caused by the neoplasia, tumor, cancer or malignancy. In some embodiments, a method of treatment results in reducing or decreasing pain, discomfort, nausea, weakness or lethargy. In some embodiments, a method of treatment results in increased energy, appetite, improved mobility or psychological well-being.

A therapeutic or beneficial effect of treatment is therefore any objective or subjective measurable or detectable improvement or benefit provided to a particular subject. A therapeutic or beneficial effect can, but need not be, complete ablation of all or any particular adverse symptom, disorder, illness, disease or complication caused by or associated with cancer pathology. Thus, treatment may be achieved when there is an incremental improvement or a partial reduction in an adverse symptom, disorder, illness, disease or complication caused by or associated with cancer pathology, or an inhibition, decrease, reduction, suppression, prevention, limit or control of worsening or progression of one or more adverse symptoms, disorders, illnesses, diseases or complications caused by or associated with cancer pathology, over a short or long duration.

A therapeutic or beneficial effect also includes reducing or eliminating the need, dosage frequency or amount of a second active treatment such as another drug or other agent (e.g., anti-neoplastic agent or treatment) used for treating a subject having or at risk of having a cancer pathology. For example, reducing an amount of an adjunct therapy, for example, a reduction or decrease of a treatment for cancer. The adjunct therapy can be a cancer treatment or therapy such as, e.g., surgery, chemotherapy, radiation therapy, or immunotherapy.

In methods, in which there is a desired outcome, such as a therapeutic or prophylactic method that provides a benefit from treatment, agonists or antagonists can be administered in a sufficient or effective amount. As used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., BNeuts), a long term or a short term detectable or measurable improvement in a given subject or any objective or subjective benefit to a given subject of any degree or for any time period or duration (e.g., for minutes, hours, days, months, years, or cured).

In some embodiments, an amount sufficient, or an amount effective, is provided in a single administration. In some embodiments, an amount sufficient, or an amount effective, is provided in multiple administrations. In some embodiments, an amount sufficient, or an amount effective, is achieved by agonists or antagonists alone, or in a composition or method that comprises a second active component. In addition, an amount sufficient or an amount effective need not be sufficient or effective if given in single or multiple doses without a second or additional administration or dosage, since additional doses, amounts or duration above and beyond such doses, or additional antigens, compounds, drugs, agents, treatment or therapeutic regimens may be included in order to provide a given subject with a detectable or measurable improvement or benefit to the subject.

An amount sufficient or an amount effective need not be therapeutically or prophylactically effective in each and every subject treated, nor a majority of subjects treated in a given group or population. An amount sufficient or an amount effective refers to the sufficiency or effectiveness in a particular subject, not a group of subjects or the general population. As is typical for such methods, different subjects will exhibit varied responses to treatment.

The term “subject” refers to an animal, typically a mammalian animal (mammal), such as a nonhuman primate (apes, gibbons, gorillas, chimpanzees, orangutans, macaques), a domestic animal (dogs and cats), a farm animal (poultry such as chickens and ducks, horses, cows, goats, sheep, pigs), experimental animal (mouse, rat, rabbit, guinea pig) and humans.

Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). Subjects include animal disease models, for example, a mouse model, and other animal models of pathogen infection known in the art. In some embodiments a mammal is a human. A mammal can be any age or at any stage of development (e.g., an adult, teen, child, infant, or a mammal in utero). A mammal can be male or female. A mammal can be a pregnant female. In certain embodiments a mammal can be an animal disease model, for example, animal models used for the study of cancer.

As used herein, a “cancer” is a disease state characterized by the presence in a subject of cells demonstrating abnormal uncontrolled replication and may be used interchangeably with the term “tumor.” In some embodiments, the cancer is a leukemia or a lymphoma. “Cell associated with the cancer” refers to those subject cells that demonstrate abnormal uncontrolled replication. In certain embodiments, the cancer is acute myeloid leukemia or acute lymphoblastic leukemia. As used herein a “leukemia” is a cancer of the blood or bone marrow characterized by an abnormal increase of immature white blood cells. The specific condition of acute myeloid leukemia (AML)—also referred to as acute myelogenous leukemia or acute myeloblastic leukemia—is a cancer of the myeloid origin blood cells, characterized by the rapid growth of abnormal myeloid cells that accumulate in the bone marrow and interfere with the production of normal blood cells. The specific condition of acute lymphoblastic leukemia (ALL)—also referred to as acute lymphocytic leukemia or acute lymphoid leukemia—is a cancer of the white blood cells, characterized by the overproduction and accumulation of malignant, immature leukocytes (lymphoblasts) resulting a lack of normal, healthy blood cells. As used herein a “lymphoma” is a cancer of the blood characterized by the development of blood cell tumors and symptoms of enlarged lymph nodes, fever, drenching sweats, unintended weight loss, itching, and constantly feeling tired.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas. Solid tumors can include, e.g., colorectal, melanoma, lung, liver, head and neck, or breast cancer.

The term “B-cell lymphoma or leukemia” refers to a type of cancer that forms in issues of the lymphatic system or bone marrow and has undergone a malignant transformation that makes the cells within the cancer pathological to the host organism with the ability to invade or spread to other parts of the body.

In some embodiments, subjects appropriate for treatment include those having or at risk of having cancer pathology.

Treatment of a cancer can be at any time during the cancer or corresponding condition. Agonists or antagonists can be administered as a combination (e.g., with a second active), or separately, concurrently or in sequence (sequentially) in accordance with the methods as a single or multiple dose e.g., one or more times hourly, daily, weekly, monthly or annually or between about 1 to 10 weeks, or for as long as appropriate, for example, to achieve a reduction in the onset, progression, severity, frequency, duration of one or more symptoms or complications associated with or caused by cancer pathology, or an adverse symptom, condition or complication associated with or caused by cancer. Thus, a method can be practiced one or more times (e.g., 1-10, 1-5 or 1-3 times) an hour, day, week, month, or year. The skilled artisan will know when it is appropriate to delay or discontinue administration. A non-limiting dosage schedule is 1-7 times per week, for 1, 2, 3, 4, 5, 6, 7, 8, 9, 10, 15, 20 or more weeks, and any numerical value or range or value within such ranges.

The exact formulation and route of administration for a composition for use according to the methods of the invention described herein can be chosen by a caregiver (e.g., a medical professional, a physician) in view of the patient's condition. See e.g., Fingl et al. 1975, in “The Pharmacological Basis of Therapeutics,” Ch. 1, p. 1; which is incorporated herein by reference in its entirety. Any suitable route of administration can be used for administration of a compound described herein. Methods of the invention may be practiced by any mode of administration or delivery, or by any route, systemic, regional and local administration or delivery. Exemplary administration and delivery routes include intravenous (i.v.), intraperitoneal (i.p.), intrarterial, intramuscular, parenteral, subcutaneous, intra-pleural, topical, dermal, intradermal, transdermal, transmucosal, intra-cranial, intra-spinal, rectal, oral (alimentary), mucosal, inhalation, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, or intralymphatic. Other non-limiting examples of routes of administration include topical or local (e.g., transdermally or cutaneously, (e.g., on the skin or epidermus), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, and the like), by parenteral administration (e.g., parenterally, e.g., intravenously, intra-arterially, intramuscularly, intraperitoneally, intradermally, subcutaneously, intracavity, intracranially, intraarticular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a composition that comprises BNeuts is provided to a subject. A composition that is provided to a subject can be provided to a subject for self-administration or to another (e.g., a caregiver, a medical professional) for administration to a subject. For example, a composition described herein can be provided as an instruction written by a medical practitioner that authorizes a patient to be provided a composition or treatment described herein (e.g., a prescription).

A dose can be administered in an effective amount or an amount sufficient to treat, prevent or slow a cancer or to treat, prevent or slow one or more adverse symptoms and/or complications of cancer. An exact dose can be determined by a caregiver or medical professional by methods known in the art (e.g., by analyzing data and/or the results of a clinical trial).

Doses can be based upon current existing protocols, empirically determined, using animal disease models or optionally in human clinical trials. Initial study doses can be based upon animal studies set forth herein, for a mouse, which weighs about 30 grams, and the amount or number of BNeut cells administered that is determined to be effective. Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. The dose can be adjusted according to the mass of a subject, and will generally be in a range from about 1 μg/kg-500 mg/kg, 1-10 μg/kg, 10-25 μg/kg, 25-50 μg/kg, 50-100 μg/kg, 100-500 μg/kg, 500-1,000 μg/kg, 1-5 mg/kg, 5-10 mg/kg, 10-20 mg/kg, 20-50 mg/kg, 50-100 mg/kg, 100-250 mg/kg, 250-500 mg/kg, or more, two, three, four, or more times per hour, day, week, month or annually. A typical range will be from about 0.3 mg/kg to about 50 mg/kg, 0-25 mg/kg, or 1.0-10 mg/kg, or any numerical value or range or value within such ranges.

Doses can vary and depend upon whether the treatment is prophylactic or therapeutic, the onset, progression, severity, frequency, duration probability of or susceptibility of the symptom, condition, pathology or complication, or immunization to which treatment is directed, the clinical endpoint desired, previous or simultaneous treatments, the general health, age, gender, race or immunological competency of the subject and other factors that will be appreciated by the skilled artisan. The skilled artisan will appreciate the factors that may influence the dosage and timing required to provide an amount sufficient for providing a therapeutic or prophylactic benefit.

Typically, for therapeutic treatment, compositions of BNeuts disclosed herein will be administered as soon as practical, typically within less than 1, 1-2, 2-4, 4-12, 12-24 or 24-72 hours after a subject is suspected of having cancer, or within less than 1, 1-2, 2-4, 4-12, 12-24 or 24-48 hours after onset or development of one or more adverse symptoms, conditions, pathologies, complications, etc., associated with or caused by cancer pathology.

BNeuts can be incorporated into compositions, including pharmaceutical compositions, e.g., a pharmaceutically acceptable carrier or excipient, such as PBS or other buffered solution with a pH of 6-8. Such pharmaceutical compositions are useful for, among other things, administration to a subject in vivo or growth of the BNeuts ex vivo followed by adoptive transfer of the cells into a patient.

As used herein the term “pharmaceutically acceptable” and “physiologically acceptable” mean a biologically acceptable formulation which is suitable for one or more routes of administration, in vivo delivery or contact. Such formulations include solutions (aqueous or non-aqueous), emulsions, suspensions, syrups, elixirs, dispersion and suspension media, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., preservatives, antibacterial, antineoplastic, antiviral and antifungal agents) can also be incorporated into the compositions, provided before, and/or after a treatment with BNeuts.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include intravenous (i.v.), inhalation, respiration, intranasal, intubation, intrapulmonary instillation, oral, buccal, intrapulmonary, intradermal, parenteral, sublingual, subcutaneous, intravascular, intrathecal, intraarticular, intracavity, transdermal, iontophoretic, intraocular, ophthalmic, optical, intramuscular, intraglandular, intraosseal, intraorgan, or intralymphatic.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include, e.g., saline, buffered solutions, or water.

Supplementary compounds (e.g., preservatives, antioxidants, antimicrobial agents including biocides and biostats such as antibacterial, antineoplastic, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include antibacterial, antiviral, antifungal and anti-parasitics. Antimicrobials include agents and compounds that kill or destroy (-cidal) or inhibit (-static) contamination by or growth, infectivity, replication, proliferation, reproduction of the microbial organism.

Exemplary anti-bacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, ribavirin, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Pharmaceutical formulations and delivery systems appropriate for the compositions and methods of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Soklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N.Y., pp. 253-315), relevant portions incorporated herein by reference.

Neutrophils are innate immune cells that are early responders to infection and inflammation. Historically, neutrophils were described as a relatively homogenous, but recent studies show that neutrophils display phenotypic and functional heterogeneity. Neutrophils have been shown to play both pro-tumoral and anti-tumoral roles but exactly which neutrophil subsets are pro-tumoral vs anti-tumoral is still unclear.

To better understand and target neutrophil functions in cancer, we must dissect neutrophil heterogeneity and how it changes from health to disease. To this end, the present inventors employed single-cell transcriptome technology to profile immature neutrophils from bone marrow of healthy individuals. Intriguingly, the inventors identified a new neutrophil subset that expresses several B cell markers, including CD79a/b, a components of the B cell receptors and PAX5, a lineage-specific transcriptional factor. The new neutrophil subset that expresses several B cell markers, including CD79a/b, a components of the B cell receptors and PAX5, a lineage-specific transcriptional factor is referred to herein as a BNeut.

Using flow cytometry, it was found that BNeuts are confined to the bone marrow of healthy individuals but expand into the blood of melanoma patients at an early stage, showing that BNeuts are biomarkers for disease. Further, it was found that BNeuts in the mouse models of breast and lung cancer. The BNeut appears and acts more like a neutrophil than B cell with a neutrophil-like transcriptional profile, large cytoplasm, ROS production, and ability to produce NETs.

Using imaging, it was also shown that BNeuts are single cells co-expressing both CD66b and CD79b, in the absence of the B cell surface marker CD19. To understand why neutrophils might use a B cell program, different B cell functions including antigen presentation, a function that is usually absent from neutrophils we determined. It was found that BNeuts are excellent at phagocytosis, including uptake of tumor cells. Further, it was found that BNeuts have increased expression of antigen presentation related genes compared to other neutrophils and can modulate CD4+ T cell responses. This newly identified novel neutrophil subset expressing B cell markers is preferentially expanded in the blood of melanoma patients and controls T cell responses during disease.

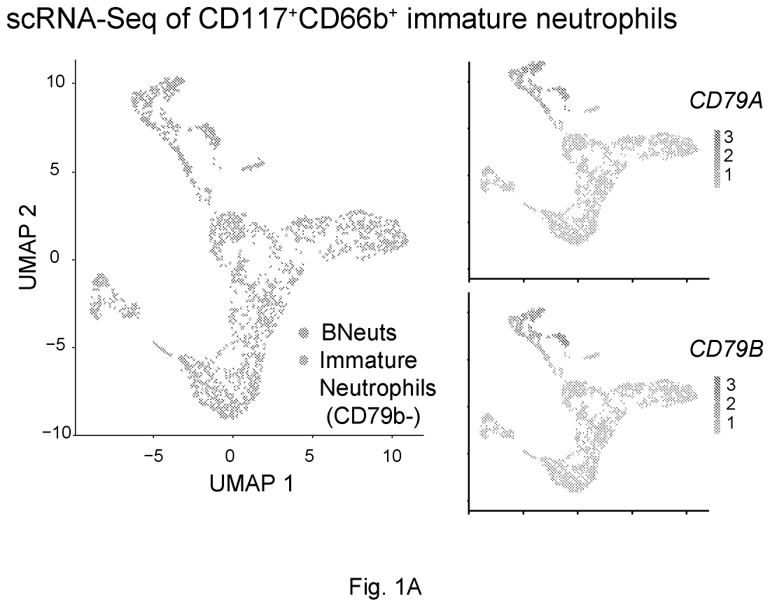

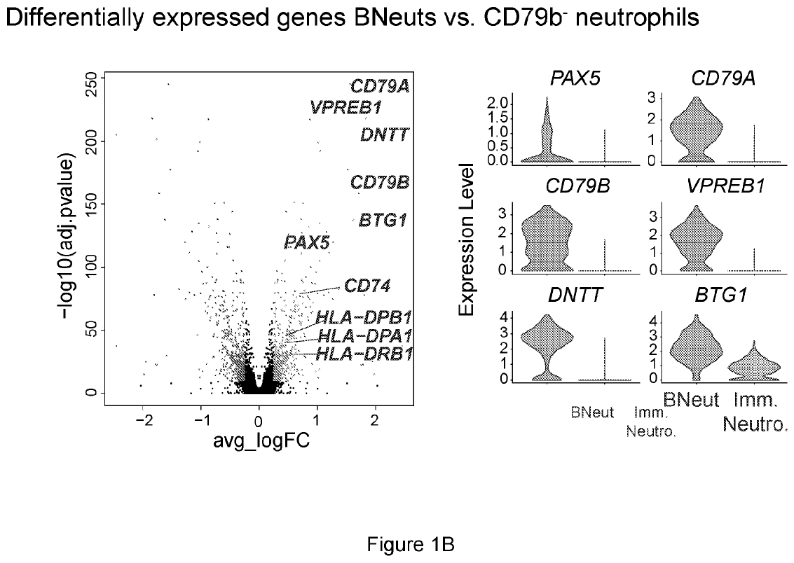

FIGS. 1A and 1B show the identification of immature neutrophils with B cell features, namely BNeuts, in healthy human bone. FIG. 1A) 10× scRNA-seq of human bone marrow Lin−CD66b+CD117+ immature neutrophils identifies a new neutrophils subset that expresses B cell genes. Cells were clustered using Louvain community (Seurat) and plotted using UMaP. FIG. 1B) Differential gene expression between BNeuts and CD79b− immature neutrophils shows increased BNeut expression of a number of B cell genes.

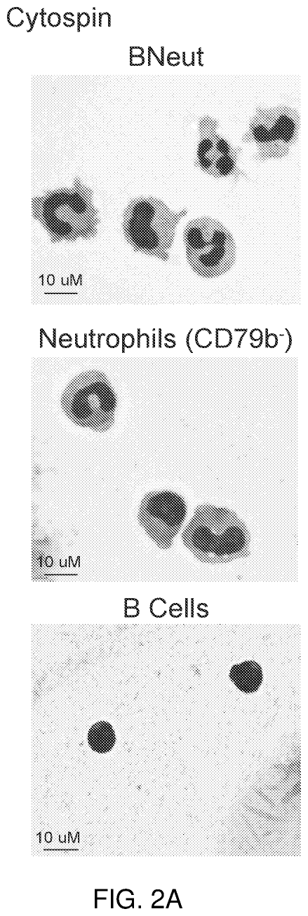

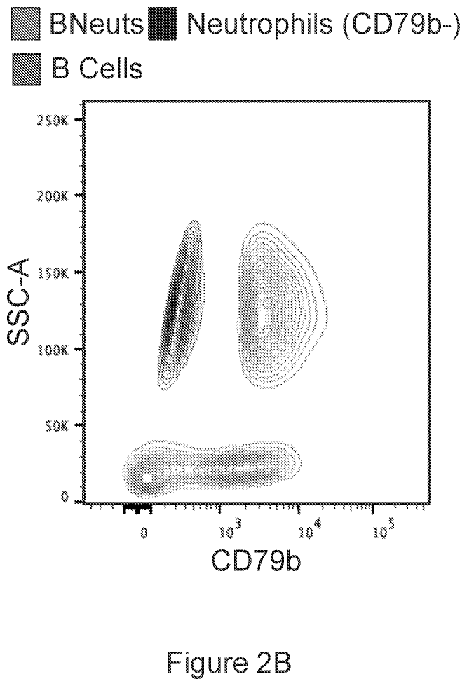

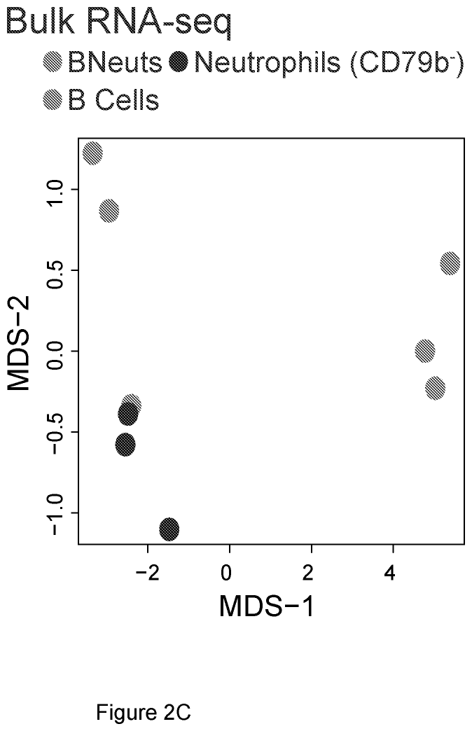

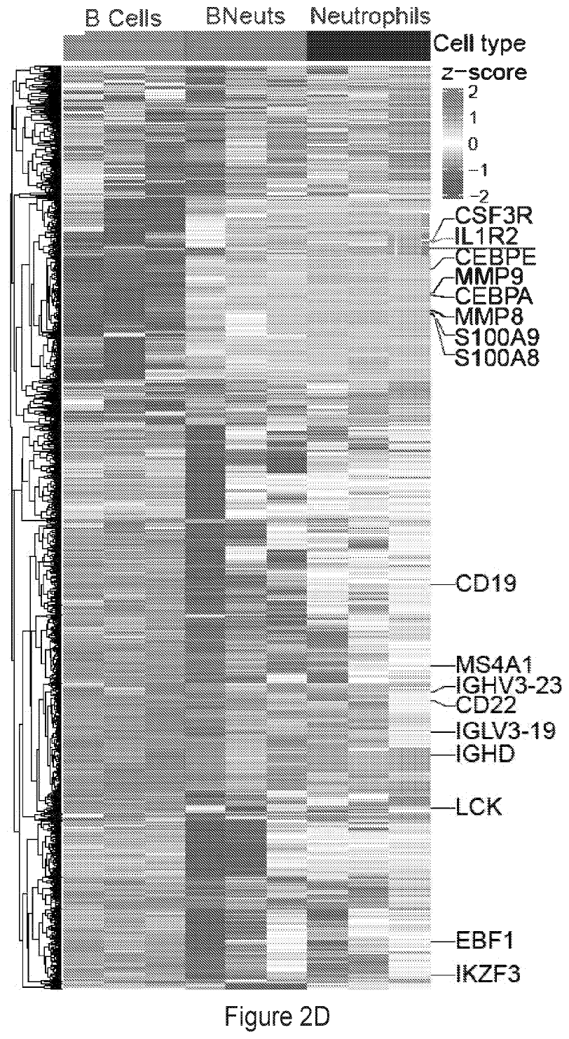

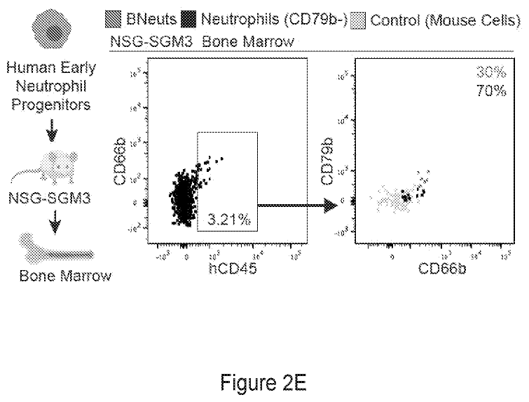

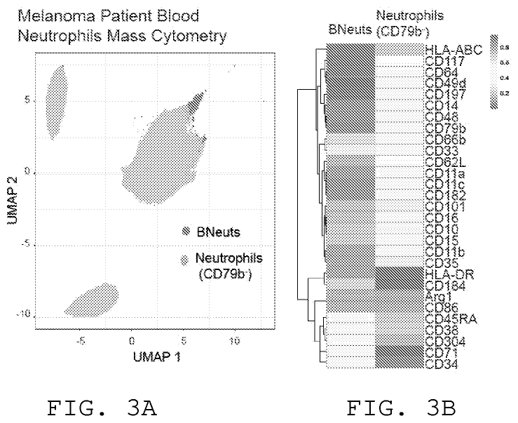

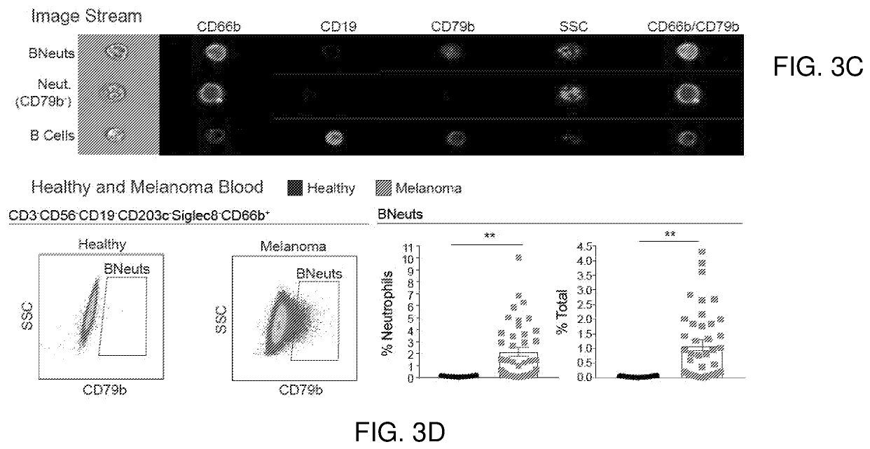

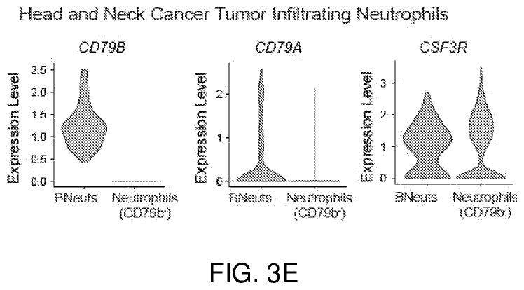

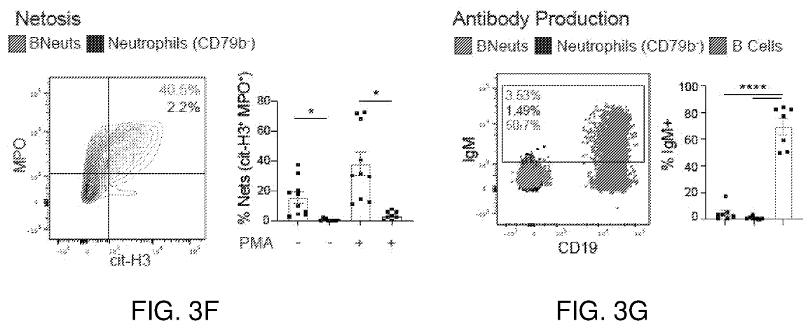

FIGS. 2A to 2E show that bone marrow BNeuts maintain neutrophil phenotypes and derive from neutrophils progenitors. Analysis of healthy human bone marrow BNeuts (see FIG. 3D), neutrophils (CD3−CD56−CD19−CD203c−Siglec8−CD66b+CD79b−), and B cells (CD3−CD56−CD66b−CD19+). FIG. 2A) Cytospins show BNeuts have a large cytoplasm, similar to neutrophils. FIG. 2B) Flow cytometry shows BNeuts maintain a high side scatter, like neutrophils, and express equivalent levels of CD79b to B cells. FIG. 2C-FIG. 2D) Bulk RNA-seq shows BNeuts cluster closer to neutrophils than B cells in MDS analysis which is reflected in the heatmap. FIG. 2E) Human early neutrophil progenitors that are restricted to the neutrophil lineage were transferred into NSG-SGM3 mice. After 5 days mouse bone marrow was analyzed for human BNeuts. CD79b+ BNeuts, indicated in red, show BNeuts can derive from neutrophil progenitors.

FIGS. 3A to 3G shows that BNeuts are found in the periphery of cancer patients and maintain neutrophil functions. Melanoma patient blood neutrophils analyzed by mass cytometry reveals CD79b+ BNeuts. FIG. 3A) Mass cytometry data projected into a UMaP. BNeuts highlighted in red identified by CD79b expression. FIG. 3B) Heatmap reveals unique protein expression pattern in BNeuts compared to neutrophils (CD79b−). FIG. 3C) Image stream of melanoma patient blood BNeuts, neutrophils (CD79b−), and B cells shows BNeuts are single cells that co-express CD79b and CD66b. FIG. 3D) BNeuts quantitation in the blood by flow cytometry reveals BNeuts are enriched in the periphery of melanoma patients. FIG. 3E) BNeuts identified in publicly available scRNA-seq data of head and neck tumor infiltrating neutrophils by CD79b and CD79a expression (Cillo et al., 2020). FIG. 3F) Netosis measured in BNeuts and neutrophils (CD79b−) from melanoma patient blood by flow cytometry. Whole blood was incubated with and without PMA. NETs identified as MPO+cit-H3+. FIG. 3G) Antibody production measured by cell surface expression of IgM (and IgD-data not shown) indicates BNeuts do not produce antibodies and present them on the cell surface. * p<0.05, ** p<0.01, **** p<0.0001 by unpaired T test.

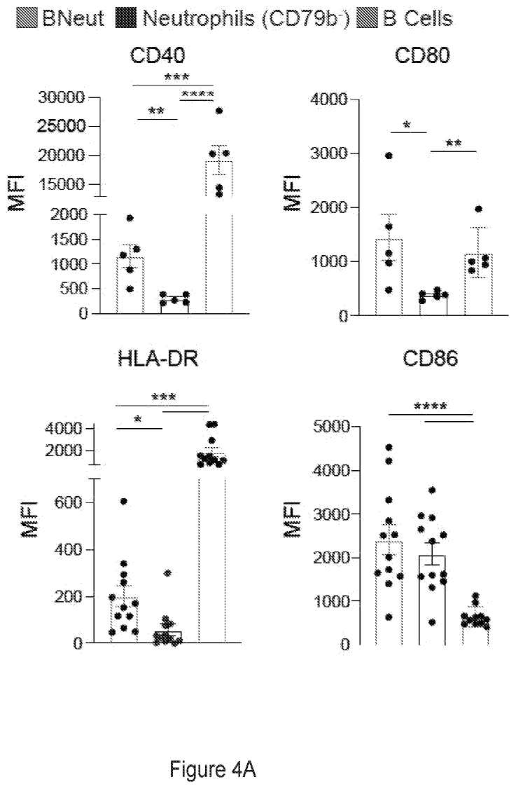

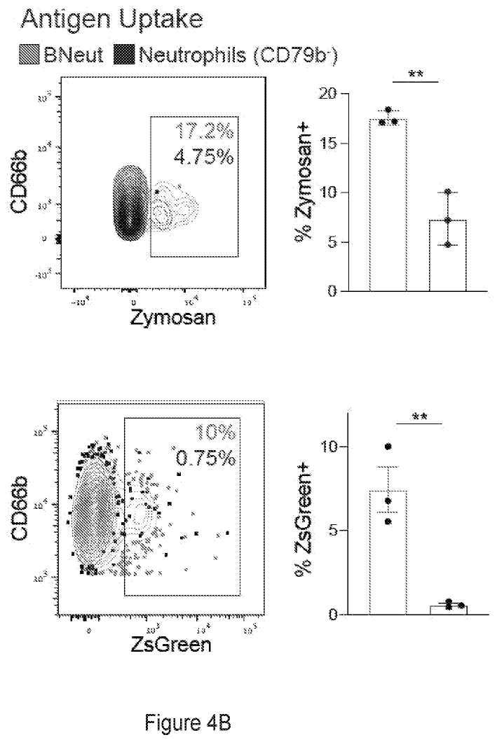

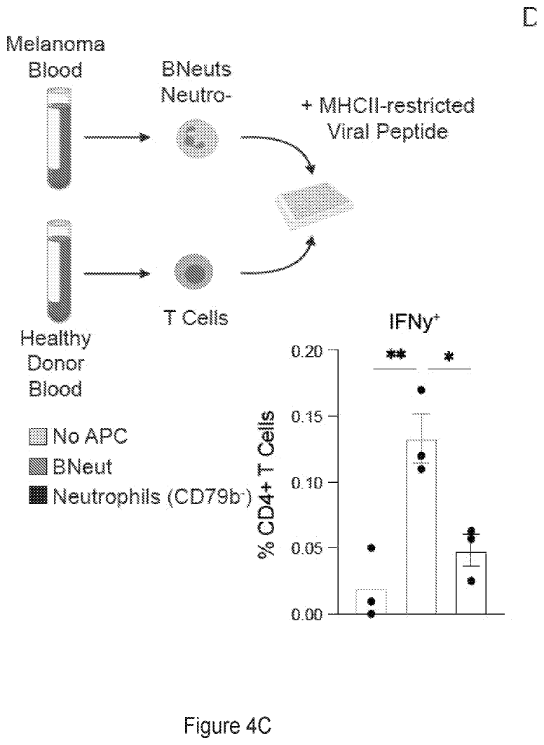

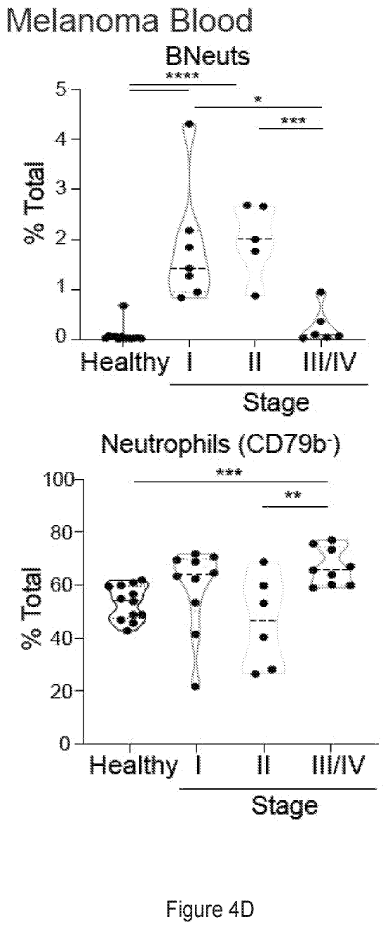

FIGS. 4A to 4D shows that BNeuts express an antigen presentation program to modulate CD4+ T Cells. FIG. 4A) Antigen presentation machinery measured by flow cytometry in BNeut, neutrophils (CD79b−), and B cells from melanoma patient blood shows elevated expression of CD40, CD80, and HLA-DR in BNeuts compared to neutrophils (CD79b−). FIG. 4B) Antigen uptake is increased in BNeuts compared to neutrophils (CD79b−) from melanoma patient blood. Whole blood was co-cultured with either zymosan (top graph) or ZsGreen+ tumor cells (bottom graph). Zymosan/ZsGreen uptake was measured by flow cytometry. FIG. 4C) BNeuts are able to stimulate CD4+ T cell viral memory responses. BNeuts or neutrophils (CD79b−) were isolated from melanoma patient blood and loaded with MHCII restricted viral peptides. After, BNeuts or neutrophils (CD79b−) were co-culture with healthy donor T cells and CD4+ T cells IFNy expression was measured by flow cytometry, as an indication of activation. FIG. 4D) BNeuts are enriched in melanoma patient blood in stage I and II. BNeuts and neutrophils (CD79b−) were measured in melanoma patient blood and healthy donors by flow cytometry. * p<0.05, ** p<0.01, *** p<0.001, **** p<0.0001 by unpaired T test.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, numerical values are often presented in a range format throughout this document. The use of a range format is merely for convenience and brevity and should not be construed as an inflexible limitation on the scope of the invention.

Accordingly, the use of a range expressly includes all possible subranges, all individual numerical values within that range, and all numerical values or numerical ranges include integers within such ranges and fractions of the values or the integers within ranges unless the context clearly indicates otherwise. This construction applies regardless of the breadth of the range and in all contexts throughout this patent document. Thus, to illustrate, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5 fold therefore includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth. Further, for example, reference to a series of ranges of 2-72 hours, 2-48 hours, 4-24 hours, 4-18 hours and 6-12 hours, includes ranges of 2-6 hours, 2, 12 hours, 2-18 hours, 2-24 hours, etc., and 4-27 hours, 4-48 hours, 4-6 hours, etc.

As also used herein a series of range formats are used throughout this document. The use of a series of ranges includes combinations of the upper and lower ranges to provide a range. Accordingly, a series of ranges include ranges which combine the values of the boundaries of different ranges within the series. This construction applies regardless of the breadth of the range and in all contexts throughout this patent document. Thus, for example, reference to a series of ranges such as 5-10, 10-20, 20-30, 30-40, 40-50, 50-75, 75-100, 100-150, and 150-171, includes ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-171, and so forth.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A method of detecting B-cell like neutrophils (“BNeuts”) comprising:
obtaining a biological sample from a subject; and
detecting whether BNeuts are present or increased in the biological sample by contacting the biological sample with one or more agents capable of detecting CD3−CD56−CD79b⁺CD66b⁺PAX5⁺CD19− cells, CD3− CD56−CD79a⁺CD79b⁺CD66b⁺PAX5⁺CD19− cells, or both.

2. The method of claim 1, further comprising detecting the presence or an elevated level of expression of at least one of: CD40, CD80, CD86, HLA-DR on the BNeuts.

3. The method of claim 1, wherein the step of detecting is measuring mRNA, protein, or both.

4. The method of claim 1, wherein the biological sample is a blood sample.

5. The method of claim 1, wherein the biological sample is obtained from a subject suspected of having cancer.

6. The method of claim 5, wherein the cancer is at an early stage.

7. The method of claim 5 or claim 6, wherein the cancer is selected from a colorectal, a melanoma, a lung, a liver, a head and neck, or a breast cancer issue.

8. The method of claim 1, wherein the presence or increase of BNeuts is detected in the biological sample when compared to an amount of BNeuts in a healthy patient.

9. The method of claim 1, wherein the one or more agents is an antibody, a small molecule, a peptide mimetic, an aptamer, a nucleic acid, or combinations thereof.

10. A method of diagnosing and treating a cancer in a patient, the method comprising the steps of:
determining whether the patient has, or has an increase of, B-cell like neutrophils (BNeut) cells by:
obtaining or having obtained a biological sample from the patient;
performing or having performed an assay on the biological sample to determine an amount of BNeuts in the biological sample, wherein the BNeuts are CD3−CD56−CD79b⁺CD66b⁺PAX5⁺CD19− cells, CD3−CD56−CD79a⁺CD79b⁺CD66b⁺PAX5⁺CD19− cells, or both;
identifying that the patient has a presence or an increase in BNeuts when compared to a reference level generated from a healthy patient; and
if the patient has BNeuts, or shows an increase in BNeuts, administering a cancer treatment or therapy to the patient.

11. The method of claim 10, wherein the presence of BNeuts is determined from a blood sample.

12. The method of claim 10, wherein the step of detecting is measuring mRNA, protein, or both.

13. The method of claim 10, wherein the cancer is an early-stage cancer.

14. The method of claim 10, wherein the cancer is selected from a colorectal, a melanoma, a lung, a liver, a head and neck, and a breast cancer.

15. The method of claim 10, further comprising detecting if the BNeuts express one or more of the following markers: CD40, CD80, CD86, or HLA-DR.

16. A method for treating a patient suffering from a cancer, the method comprising the steps of:
determining whether the patient has B-cell like neutrophils (BNeut) or an increase in BNeuts, when compared to a reference level generated for a healthy patient by:
obtaining or having obtained a biological sample from the patient; and
performing or having performed an assay on the biological sample to determine if the patient has BNeuts; and
if the patient has BNeuts, then administering a cancer treatment or therapy to the patient.

17. The method of claim 16, wherein the presence of BNeuts is determined from blood sample.

18. The method of claim 16, wherein the step of detecting is measuring mRNA, protein, or both.

19. The method of claim 16, wherein the BNeuts are CD3− CD56−CD79b⁺CD66b⁺PAX5⁺CD19− cells, CD3− CD56−CD79a⁺CD79b⁺CD66b⁺PAX5⁺CD19− cells or both.

20. The method of claim 16, wherein the cancer is an early-stage cancer.

21. The method of claim 16, wherein the cancer is selected from a colorectal, a melanoma, a lung, a liver, a head and neck, and a breast cancer.

22. The method of claim 16, wherein the BNeuts express or have an elevated level of expression one or more of the following markers: CD40, CD80, CD86, or HLA-DR.

23. The method of claim 10 or claim 16, wherein the cancer treatment or therapy comprises surgery, chemotherapy, radiation therapy, or cancer immunotherapy.

24. The method of claim 23, wherein the cancer immunotherapy comprises adoptive cell therapy.

25. The method of claim 24, wherein the adoptive cell therapy comprises administering a population of BNeuts, CD3− CD56−CD79b⁺CD66b⁺PAX5⁺CD19− cells, CD3−CD56−CD79a⁺CD79b⁺CD66b⁺PAX5⁺CD19− cells, or any combination thereof.

26. The method of claim 24 or claim 25, wherein the adoptive cell therapy comprises administering a population of engineered cells.

27. A method for treating a cancer patient, the method comprising the steps of:
determining whether the cancer patient has an increase in B-cell like neutrophils (“BNeuts”) by:
obtaining or having obtained a biological sample from the patient;
performing or having performed an assay on the biological sample to determine if the patient has an increase in BNeuts when compared to a baseline level of BNeuts present in a healthy patient; and
if the patient has an increase in BNeuts when compared to a baseline level of BNeuts present in the healthy patient, then providing an adoptive cell therapy that comprises BNeuts, CD3−CD56−CD79b⁺CD66b⁺PAX5⁺CD19− cells, CD3−CD56−CD79a⁺CD79b⁺CD66b⁺PAX5⁺CD19− cells, or any combination thereof, to the patient in an amount sufficient to reduce or treat the cancer.

28. The method of claim 27, wherein the cancer is an early-stage cancer.

29. The method of claim 27, wherein the cancer is selected from a colorectal, a melanoma, a lung, a liver, a head and neck, and a breast cancer.

30. The method of claim 27, further comprising the step of isolating BNeuts from the cancer patient, growing the BNeuts ex vivo, and isolating the BNeuts prior to the adoptive cell therapy.

31. The method of claim 27, wherein the BNeuts are autologous, syngeneic, allogeneic, or xenogeneic.

32. A kit for cell sorting or detecting B-cell like neutrophils (“BNeuts”) from a biological sample, the kit comprising detecting agents for at least one of: CD3−CD56−CD79b⁺CD66b⁺PAX5⁺CD19− cells, CD3− CD56−CD79a⁺CD79b⁺CD66b⁺PAX5⁺CD19− cells, or both.

33. A method for treating a patient, the method comprising the steps of:
identifying that the patient is in need of a treatment for cancer; and
providing an adoptive cell therapy that comprises BNeuts, CD3−CD56−CD79b⁺CD66b⁺PAX5⁺CD19− cells, CD3−CD56−CD79a⁺CD79b⁺CD66b⁺PAX5⁺CD19− cells, or any combination thereof, to the patient in an amount sufficient to reduce or treat the cancer, wherein the BNeuts are autologous, syngeneic, allogeneic, or xenogeneic.
